(19) United States
(12) Patent Application Publication
(10) Pub. No.: US 2014/0012221 A1
(43) Pub. Date: Jan. 9, 2014

(54) TAMPONS

(71) Applicant: Margaret Imelda Henson, Hertfordshire (GB)

(72) Inventor: Margaret Imelda Henson, Hertfordshire (GB)

(21) Appl. No.: 13/726,104

(22) Filed: Dec. 22, 2012

(30) Foreign Application Priority Data
Jun. 7, 2012 (GB) GB1210076.4

(51) Int. Cl. A61F 13/20

(52) U.S. Cl. CPC A61F 13/2031
USPC 604/385.17

(57) ABSTRACT

The present invention provides a tampon adapted for use by women who experience high viscosity periods/clotted menstrual blood flow. The tampon has a body comprising an elongate mass of compressed absorbent material with a proximal end (end that is inserted first) and a distal end and wherein the proximal end/tip of the tampon is formed substantially enlarged having a greater lateral spread than the distal end of the tampon prior to wetting, and has at least one of a crater, concavity, void, open hollow, cleft or lateral crevices at the proximal end to capture and hold viscous/gelatinous menstrual fluid.

### CROSS-REFERENCE TO RELATED APPLICATIONS

This application claims priority to UK Patent application GB1210076 4 filed on June 2012, the entirety of which is incorporated herein by reference.

### BACKGROUND OF THE INVENTION

1. Field of the Invention

The present invention concerns catamenial tampons.

2. Background

Worldwide the feminine hygiene and sanitary protection products industry including catamenial tampons is a multibillion dollar industry. In the UK alone, for example, the tampon market is worth about 100 million pounds a year. The technology of tampon design and construction receives considerable investment by the major corporations such as P&G (formerly Procter & Gamble), Playtex (Energizer Holdings), Lillets and Kimberley Clark, all competing to improve the product experience and gain market share.

A major selling point and target for technical design of tampons is, of course, optimal efficacy of absorption of the menstrual blood with the tampon to prevent any leakage and yet being comfortable to insert and wear and not restricting the user's freedom. Current tampons are generally marketed for trapping menstrual flows at ever increasing absorbencies, but are not designed for handling mixed viscosity flows including low viscosity blood and high viscosity blood clots. The shape of most modern tampons is dictated by these criteria and takes a broadly streamlined bullet-shaped form, being a smooth sided uniform diameter cylindrical form with a domed tip at the proximal end of the tampon that is inserted first into the vagina and having a draw-string at the distal and of the tampon for ease of subsequent withdrawal for disposal.

To assist comfortable insertion of modern tampons not only is the tampon itself streamlined but an applicator is often provided, normally comprising a pair of concentric telescoping tubes that enshroud the tampon during insertion of the applicator. When the applicator is inserted it is operated by pushing the inner tube inside the outer, which serves to propel the tampon out of the applicator into position in the vagina. Once the tampon is inserted it is designed to stay in place and absorb and hold menstrual fluid to prevent it leaking from the vagina. A tampon usually expands either width-ways or lengthways as fluid is absorbed.

In pursuing the goal of effective protection against leakage a number of modern designs of tampon have gone further to incorporate a hollow core construction of the tampon, some hollow only at the distal end for housing absorbed menstrual flow but some hollow at the proximal end and which flares open on moistening to present a central cavity/recess for capture of the menstrual fluid. Examples of such latter construction are disclosed in U.S. Pat. No. 4,335,720 (Glassman) and in U.S. Pat. No. 5,659,934 (Kimberley Clark).

As women approach the menopause, it is common for them to experience gelatinous clots in the menstrual fluid. Clots can also occur in younger women, Sometimes they are a result of medical conditions (eg: fibroids) or the fitting of an IUD device or taking the contraceptive pill. The manifestation of clots can severely interfere with the proper functioning of conventional tampons. In particular, gelatinous clots can simply slide over the convex tampon tip/proximal inserted end and pass down the sides around the tampon and leak out of the vagina because they are too thick to be absorbed by the smooth, compressed material. Also, the clots can stick to the side of the tampon which causes uneven absorption of the less viscous menstrual fluids, causing the tampon to leak before it is fully saturated.

Whereas hollow core tampons such as the patented tampons referred to above may be more effective at trapping clots than the original ‘solid’ bodied compacted fibre tampons they are still streamlined/bullet-shaped forms until deployed and are dependent upon high water content in the menstrual fluid to be properly deployed. They are not designed to address clots and do not fundamentally solve the problems.

Indeed at present the market for catamenial products aimed at least in part at pen-menopausal women or those with the afore-mentioned symptoms is dominated by devices that are not tampons at all but rather are costly to purchase and bulky moulded silicone cups that collect rather than absorb menstrual fluids and are designed to be worn much lower in the vaginal tract and designed to be reused. These present a solution to the problem but for many women they are not ideal as they are not comfortable or discreet, are too messy and require too much maintenance.

It is an object of the present invention to provide a tampon that addresses the problems described above

### SUMMARY OF THE INVENTION

The summary of the invention is provided as a general introduction to some of the embodiments of the invention, and is not intended to be limiting. Additional example embodiments including variations and alternative configurations of the invention are provided herein.

According to a first aspect of the present invention there is provided a tampon adapted for use by women who suffer from clotted menstrual blood flow, wherein the tampon has a body comprising an elongate mass of compressed/moulded absorbent material with a proximal end (end that inserts first into a user's body) and a distal end and wherein the proximal end of the tampon is formed substantially enlarged with a greater lateral spread than the distal end of the tampon (in its compressed state) prior to wetting, and has at least one longitudinal cleft and/or has a crater or concavity at the proximal end that deepens as a void or hollow core through the tampon to capture and hold viscous/high viscosity or gelatinous menstrual fluid, the tampon being absorbent through its exterior/external lateral surfaces.

Unlike conventional tampons, the tampon of the present invention foregoes having a streamlined/bullet-shaped profile for ease of insertion and that flares only after wetting. Instead the tampon of the present invention is pre-formed to have a laterally enlarged or flared proximal end/head that is immediately ready to capture high viscosity menstrual material. The proximal end of the tampon is the end of the tampon that is inserted in the user first, while the opposing distal end is the end of the tampon that commonly has a drawstring attached for retraction of the tampon.

The form of the tampon overcomes the primary problem of non-deployment/inadequate deployment of the conventional tampon in use when the periods are relatively dry or the clot precedes low viscosity blood flow on which most tampons rely for expansion. It is unpredictable when a clot will arrive. It could be there moments after inserting a fresh tampon so the tampon is likely to be dry or could arrive sometime after the lighter fluids have been absorbed. In either event, if a tampon has just been removed, it is likely that the vagina will be relatively dry when the new one is inserted.

The tampon is preferably provided with a hollow core. The proximal end concavity may resemble a crater The concavity is preferably substantially central of the proximal end and opens into/deepens into the hollow core of the tampon body. The hollow core or longitudinal cleft(s) in the side(s) of the tampon body preferably extend for the majority of the length of the body and may extend the entirety of the length of the body.

The tampon's body may be of substantially uniform lateral spread/diameter for the majority of its length but enlarged at the proximal end or more preferably it may be laterally outwardly tapered for the majority of its length leading towards the proximal end. It is suitably approximately/substantially cylindrical or, where tapered, it is preferably substantially conical or frusto-conical in form. It may, for example, be approximately/substantially circular -cylindrical, -conical or -frusta-conical in form (circular in transverse section) or ovoid -cylindrical, -conical or -frusto-conical in form (ovoid in transverse section).

The enlarged proximal end of the tampon suitably is of enlarged diameter/lateral spread not only relative to the distal end of the tampon's body but also relative to the rest of the tampon's body.

As a particularly preferred further enhancement to the tampon it preferably has at the proximal end/tip a roughened fibrous surface. The compressed absorbent fibres forming the tampon, that normally present a sleek, smooth exterior to the tampon, suitably, at the proximal end of the tampon, are cut, combed or mechanically (or otherwise) disrupted to present a carpet of fibre ends to which the blood clots/gelatinous menstrual materials are more likely to cling and thus be trapped at the proximal end of the tampon.

Particularly preferably at least the external surface of the tampon distal end (eg the crater/concavity) has microfibers and in the preferred arrangements the tampon is formed throughout from a mix of fibres and microfibers. The fibres may be the conventional cotton, rayon or cotton/rayon mix of fibres, for example. The microfibers may be present as a relatively low percentage, suitably of the order of from 1% to 30% of the fibres forming the tampon, and still be effective. The use of microfibers can increase the effectiveness of the tampon capturing menstrual materials.

The tampon body of compressed absorbent material may be a one-piece/unitary body or an assembly of parts. The body may be formed as a compacted body into which a distal end concavity is subsequently formed by a press or other implement or may be moulded directly into a shape having the concavity. Where the tampon body is formed as an assembly of parts it may for example comprise a plurality of panels that are stitched, adhered or otherwise secured together.

In various embodiments, whether a one-piece/unitary body or an assembly of parts, the tampon body as formed/completed may resemble a partially open flower, with petals that slightly spread/separate towards the top of the flower/proximal end of the tampon body. The body may be cruciform as viewed from the distal end elevation or have any number of lobes/petals.

The shapes and ways in which an assembly of separate parts/lobes may be assembled together to the final form are diverse, They may, for example, be panels that are overlaid in a stack each at a different radial orientation to a neighbouring panel and initially resembling a windmill that is then folded forwardly and the panels held together in the final form, eg to resemble a part open flower. In another arrangement the tampon body is formed by rolling a panel of compressed absorbent material around itself. The panel may have an inclined top edge or the panel may be asymmetric being shorter at one end edge that becomes enrolled at the core of the body and thereby defines a concavity at the core of the body.

In another embodiment the tampon body is formed by placing two panels of compressed absorbent material back to back and stitching or otherwise securing them together at a central axis whereby the tampon body has a cruciform shape (X-shape) as viewed in proximal end elevation.

In view of the large head/proximally enlarged shape of the tampon it suitably is partnered with a tampon applicator for comfortable insertion, the applicator suitably being of uniform external width/diameter. The applicator internal chamber width/diameter is sized to accommodate the large head/proximally enlarged shape of the tampon proximal end and has a convex rounded tip/leading end that houses the enlarged concave proximal end of the tampon body. Where the internal chamber of the applicator is of uniform broad diameter and the distal end of the tampon body is relatively narrow, a centraliser guide may be provided in the applicator to keep the tampon body axially aligned with the applicator for smooth ejection. The centraliser is suitably formed at the leading pusher/plunger part of the applicator.

The invention as described and illustrated herein provides a much more reliable solution to the problems of the prior tampons.

### BRIEF DESCRIPTION OF THE DRAWINGS

Preferred embodiments of the invention will now be more particularly described, solely by way of example, with reference to the accompanying drawings in which:

### DETAILED DESCRIPTION OF THE ILLUSTRATED EMBODIMENTS

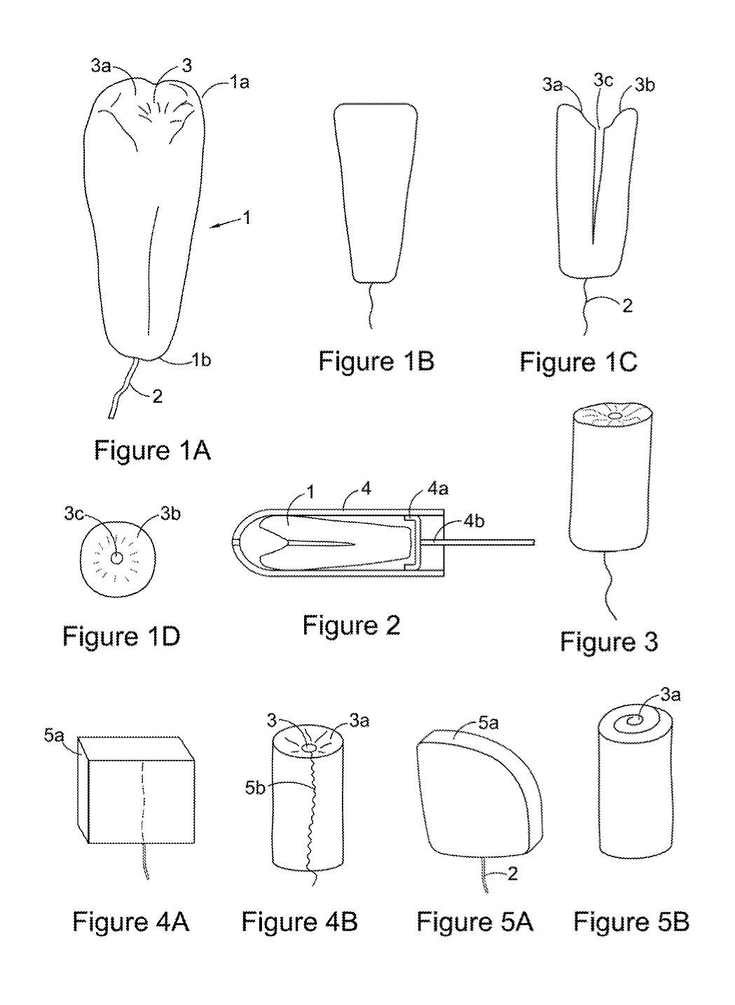

FIG. 1A is a perspective view of a first embodiment of the invention, while FIG. 1B is a side elevation view thereof; FIG. 1C is a long sectional view thereof; and FIG. 1D is an end elevation view of the proximal end thereof;

FIG. 2 is a longitudinal sectional view of a streamlined tampon applicator housing the tampon body prior to deployment for ease of initial insertion;

FIG. 3 is a perspective view of a circular cylindrical form of the absorbent material before compressing/moulding into the tapered/conical shape;

FIG. 4A is a perspective view of a single absorbent fibrous panel that may be furled to form the tampon body and FIG. 4B is a perspective view of that single absorbent fibrous panel as furled;

FIG. 5A is a perspective view of a single absorbent fibrous panel that may be rolled up like a Swiss roll to form the tampon body and FIG. 5B is a perspective view of that fibrous panel as rolled up;

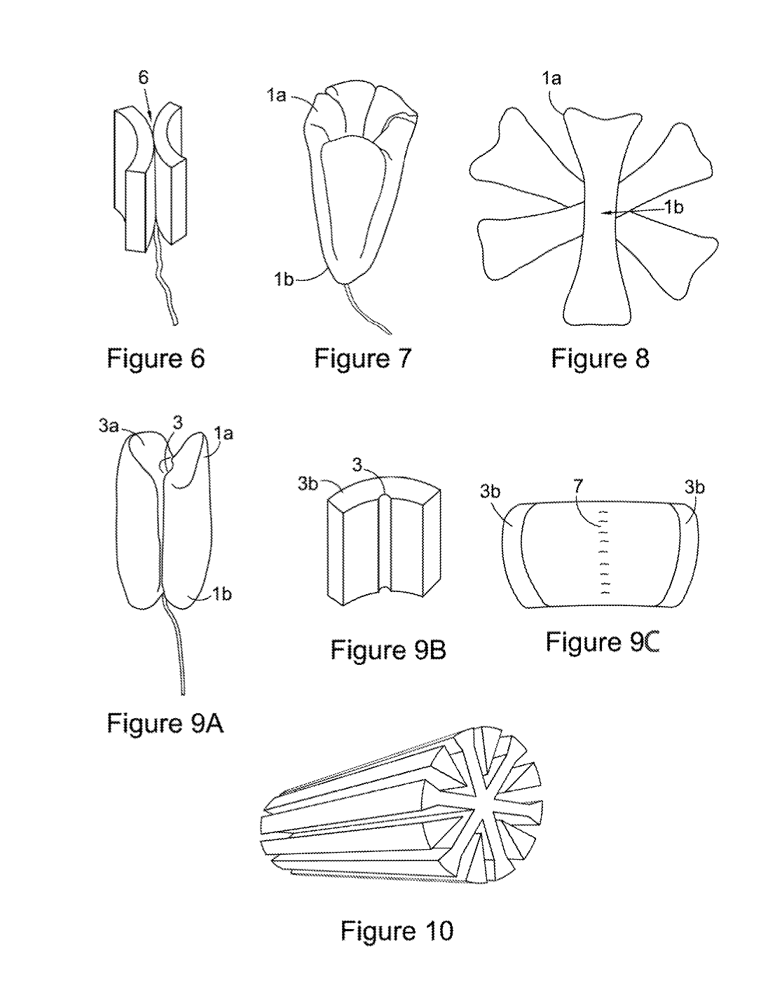

FIG. 6 is a perspective view of an embodiment of the invention formed as an assembly of two panels of absorbent material secured together at the longitudinal axis of the body (in this image the tampon body is shown prior to compression into the required proximally laterally outwardly tapered form);

FIG. 7 is a perspective view of an embodiment of the invention formed as single body of absorbent material having multiple lobes and resembling a flower;

FIG. 8 is a perspective view of a multi-panel approach to assembling and forming the multi-lobed/flower shaped form of tampon body;

FIG. 9A is a perspective view of a two-lobed form of tampon while FIG. 9B is a perspective view of one panel of a pair that are assembled together facing each other to form the two-lobed body and FIG. 9C is a perspective view of an alternative forming method for the two-lobed tampon body comprising a single panel that is to be folded about a median line to form the two-lobed tampon body; and

FIG. 10 is a figurative schematic of a typical microfiber.

### DESCRIPTION OF THE PREFERRED EMBODIMENTS

Corresponding reference characters indicate corresponding parts throughout the several views of the figures. The figures represent an illustration of some of the embodiments of the present invention and are not to be construed as limiting the scope of the invention in any manner. Further, the figures are not necessarily to scale, some features may be exaggerated to show details of particular components. Therefore, specific structural and functional details disclosed herein are not to be interpreted as limiting, but merely as a representative basis for teaching one skilled in the art to variously employ the present invention

As used herein, the terms “comprises,” “comprising,” “includes.” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus, Also, use of “a” or “an” are employed to describe elements and components described herein. This is done merely for convenience and to give a general sense of the scope of the invention. This description should be read to include one or at least one and the singular also includes the plural unless it is obvious that it is meant otherwise.

Certain exemplary embodiments of the present invention are described herein and illustrated in the accompanying figures. The embodiments described are only for purposes of illustrating the present invention and should not be interpreted as limiting the scope of the invention. Other embodiments of the invention, and certain modifications, combinations and improvements of the described embodiments, will occur to those skilled in the art and all such alternate embodiments, combinations, modifications, improvements are within the scope of the present invention.

Referring to FIGS. 1A-D, the first illustrated embodiment of the tampon has a tampon body 1 that is of unitary construction, being compressed and moulded from a block of loose fibrous absorbent material, such as of a mix of cotton and rayon fibres. The mix suitably further comprises 10% by mass of microfibers dispersed throughout or applied at the outer surface of the block. The shape of the body 1 is substantially circular cylindrical but tapers laterally/radially outwardly from a distal end 1b of the tampon body 1 to a comparatively broader proximal end 1a of the tampon body. The proximal end of the tampon body 1 is the leading end when being inserted by the user for use while the distal end lb has a drawstring 2 extending out therefrom as a means of withdrawing the tampon after use. The provision of such a drawstring 2 is very useful, given the shape of the tampon body 1, and should generally be included in all variants and embodiments of the invention.

The broader proximal end 1a resembles a crater, having a central concavity 3a with a raised rim 3b. At the centre of the concavity 3a it deepens/opens downwardly into a hollow core 3c of the tampon body 1 that can serve for trapping and anchoring/rooting clots/gelatinous material. The overall shape/form of the tampon body may be determined in a number of different ways that will be discussed later, in this first embodiment it is compressed and moulded from a single cylindrical block of the loose fibrous absorbent material, such as is shown in FIG. 3. The hollow core 3c extends to ⅔ or more the length of the tampon body 1.

The surface of the proximal end la at the concavity 3a and rim 3b may be cut, brushed, or otherwise treated to roughen it, ensuring that there are many fibre ends projecting from the surface. The end surface is may have this form through a roughening treatment step that is applied post-compression of the absorbent tampon material into the required tampon body shape or, in some embodiments, it may be created from the absorbent tampon material by the manner in which the tampon body is formed/assembled, eg by ensuring that the cut end of a substantially homogeneously aligned mass of fibres is presented as the proximal end of the tampon body 1. The relative roughness of the proximal end is and the microfibers in the tampon body 1 provide improved capture of the menstrual fluid/materials. From FIG. 10 it can be seen that a typical microfiber has micro-crevices in its external faces that increase its surface area and may increase the likelihood of adhesion of any clot.

The tampon body 1 is suitably substantially frusto-conical in form as moulded, and has a substantially trapezoidal shape as viewed in side elevation, as can be seen in FIG. 1B, The tampon body 1 has a broad proximal end 1a to provide a landing area of sufficient size for the blood clots to reliably contact it/land onto it. The body 1 broadens/tapers outwardly from the narrower distal end 1b to the broader proximal end 1a. When viewed from above down onto the proximal end 1a of the tampon body 1 it suitably is circular as seen in FIG. 1D or oval in shape.

The external side surface/conic or circumferential surface of the body 1 is absorbent to allow fluids to be absorbed by the tampon. There is no impervious membrane/sheath or other moisture barrier around the tampon body 1 and much of the absorption of free-flowing, low viscosity blood can thus be at the sidewall—which is especially useful when the proximal end la of the tampon body 1 is at least partly covered/occluded by a clot.

The tampon body 1 is partnered with a tampon applicator 4, as shown in FIG. 2, for comfortable insertion, The applicator 4 is of uniform external width/diameter while the applicators internal chamber width/diameter is sized to accommodate the large proximal end la of the tampon body land has a convex rounded tip/leading end that houses the tampon body proximal end 1a. Since the internal chamber of the applicator 4 is of uniform broad diameter and the distal end of the tampon body la is relatively narrow, a centraliser guide 4a is provided at the leading pusher plunger part 4b of the applicator 4 to keep the tampon body 1 axially aligned with the applicator 4 for smooth ejection.

In basic variations of the first embodiment of tampon body 1 the end elevation/transverse sectional shape of the tampon body 1 may be made oval rather than circular, or may be multi-lobed as will be described later. The method of manufacture of the tampon body 1 may be one of a number of different methods. Although described and illustrated with respect to FIGS. 1A-D and 3 as being a direct moulding from a single solid circular cylindrical fibrous mass, it may for example alternatively be formed from a flat panel/pad of the absorbent fibrous material that is flexed in a curve to form a hollow circular cylinder (FIG. 4B) or may be further rolled to resemble a Swiss roll (FIG. 58) before being compressed to the final shape. The Swiss roll form may rolled around a removable core former to ensure there remains a hollow core at the heart of the body 1.

In the case of the FIG. 4B variant the panel/pad may be pre-formed to have an inclined upper end edge 5a, inclined across the thickness of the panel/pad as shown in FIG. 4A, thus pre-defining the concavity 3a when the pad/panel is furled rather than requiring subsequent compression moulding to form the concavity 3a. It may then be secured in the cylindrical shape and configuration by stitching or by adhesive at the mating longitudinal panel/pad edges 5b.Furthermore, the panel/pad may be pre-compressed to a pre-tapered form in cross section prior to furling into the end shape and may again be secured in the conical shape and configuration by stitching or by adhesive at the mating longitudinal panel/pad edges 5b.

In the case of the FIG. 5B variant the panel/pad may be pre-formed to have an arcing upper end edge 5a down-curving along the length of the pad/panel as shown in FIG. 5A, thus pre-defining the concavity 3a when the pad/panel is furled with the lowered end of the arcing upper end edge 5a being innermost/at the centre of the body 1 when the pad/panel is furled.

The body may in some variants lack a hollow core and simply have a concavity/crater at the proximal end. This latter configuration is most appropriate if the sidewalls of the tampon body define crevices/slits/clefts in which the clot may be trapped. In such a variant embodiment, illustrated in FIG. 6, the tampon body 1 is formed as an assembly of two panels/pads of fibrous absorbent material placed back to back and secured together by stitching or adhesive along a vertical axial line 6 about which each is folded out to define a cruciform shape. We have found that having a split assembly or multi-lobed configuration construction of the tampon body, such as with this cruciform tampon body, can be advantageous in enhancing efficacy. The crevices/formed by the opposing/contacting panel/pad faces provide clefts in which the clot can become trapped, helping to anchor it to the top of the tampon. Also the increased surface area for contact and for absorption provided by the crevices substantially improves the swift absorption of the less viscous menstrual fluids.

Other mufti-lobed variants of the tampon body 1 are illustrated in FIGS. 5 A to 9C and variously comprise a flower-like multi-lobed tampon body 1 as illustrated in FIGS. 7 and 8 and which may be of unitary moulded construction as in FIG. 7, Alternatively they may be assembled from multiple panels that are stacked on each other at differing orientations, like a windmill as shown in FIG. 8, and then folded up and secured in a flower-like conformation as per FIG. 7. The lowermost panels in the stack which become outermost as they are folded up to the flower-like conformation are longer than the uppermost to compensate for the shortening effect as they are folded upwardly and to ensure that the final shape is concave at the proximal end. A simpler two-lobed variant of the multi-lobed tampon body is shown in FIG. 9A-9C and may be made as a one-piece body that is formed from a single shaped panel that is folded up about a transverse base axis 7. Alternatively it may be assembled from a pair of panels like the one shown in FIG. 9B, with the pair being mated facing each other and secured in the conformation of FIG. 9A. A hollow core is suitably still preserved in the FIG. 9A embodiment as can be seen at the proximal end la in FIG. 9A.

Numerous other embodiments and variants are conceivable within the sprit and scope of the invention and are embraced within the invention as set forth in the description and drawings and claims hereinafter

It will be apparent to those skilled in the art that various modifications, combinations and variations can be made in the present invention without departing from the spirit or scope of the invention. Specific embodiments, features and elements described herein may be modified, and/or combined in any suitable manner. Thus, it is intended that the present invention cover the modifications, combinations and variations of this invention provided they come within the scope of the appended claims and their equivalents.

I claim:

1. A tampon adapted for use by women who suffer from clotted menstrual blood flow, wherein the tampon has a body comprising an elongate mass of compressed absorbent material with a proximal end and a distal end and wherein the proximal end of the tampon is formed substantially enlarged with a greater lateral spread than the distal end of the tampon prior to wetting, and has at least one longitudinal cleft in its side and/or a crater or concavity at the proximal end that deepens as a void or hollow core through the tampon to capture and hold viscous or gelatinous menstrual fluid, the tampon being absorbent through its exterior surfaces.

2. An adapted tampon as claimed in claim 1, wherein the cleft or void or hollow core, extends for at least a quarter of the length of the tampon.

3. A tampon as claimed in claim 2, wherein the cleft or void or hollow core extends for the entire length of the tampon.

4. A tampon as claimed in claim 1, wherein the tampon enlarged proximal end extends towards the distal end for at least a quarter of the length of the tampon.

5. A tampon as claimed in claim 1, wherein the tampon's body is laterally outwardly tapered leading towards the proximal end.

6. A tampon as claimed in claim 5, wherein the tampon's body is substantially frusto-conical in form, laterally outwardly tapered leading towards the proximal end and/or has a substantially trapezoidal shape as viewed in side elevation.

7. A tampon as claimed in claim 1, wherein the proximal end has a concavity that forms a crater that is substantially central of the proximal end.

8. A tampon as claimed in claim 1, wherein the tampon's body is substantial cylindrical but with an enlarged width/radius proximal end.

9. A tampon as claimed in claim 1, wherein at least the external surface of the tampon proximal end at the crater or concavity has microfibers.

10. A tampon as claimed in claim 1, wherein the tampon body of compressed absorbent material is a one-piece/unitary body formed as a compacted body into which a proximal end crater or concavity is subsequently formed by a press or other implement or the material is moulded directly into a shape having the crater or concavity.

11. A tampon as claimed in claim 1, wherein the tampon body is formed by roiling a panel of compressed absorbent material around itself.

12. A tampon as claimed in claim 11, wherein the tampon body is formed by rolling a panel of compressed absorbent material around itself wherein the panel is asymmetric being shorter at one end edge that becomes enrolled at the core of the body and thereby defines a concavity at the core of the body.

13. A tampon as claimed in claim 1, wherein the tampon body is formed by placing two panels of compressed absorbent material back to back and stitching or otherwise securing them together at a central axis whereby the tampon body has a cruciform shape as viewed in proximal end elevation

14. A tampon adapted for use by women who suffer from clotted menstrual blood flow, wherein the tampon has a body comprising an elongate mass of compressed absorbent material with a proximal end and a distal end and wherein the proximal end of the tampon is formed substantially enlarged with a greater lateral spread than the distal end of the tampon prior to wetting, and has a crater, concavity or opening at the proximal end to capture and hold viscous or gelatinous menstrual fluid, wherein the tampon has at the proximal end a roughened fibrous surface.

15. A tampon as claimed in claim 14, wherein the compressed absorbent fibres forming the tampon are at the proximal end of the tampon cut, combed/mechanically (or otherwise) disrupted to present a carpet of loose fibres by which the blood clots/tissues tissue/gelatinous menstrual materials are better trapped.

16. A tampon as claimed in claim 14, wherein at least the external surface of the tampon proximal end at the crater or concavity has microfibers.

17. A tampon adapted for use by women who suffer from clotted menstrual blood flow, wherein the tampon has a body comprising an elongate mass of compressed absorbent material with a proximal end and a distal end and wherein the proximal end of the tampon is formed substantially enlarged with a greater lateral spread than the distal end of the tampon prior to wetting, and has a longitudinal cleft in a side and/or a crater or concavity at the proximal end, wherein the tampon body of compressed absorbent material is an assembly of parts that comprise a plurality of panels that are stitched, adhered or otherwise secured together.

18. A tampon as claimed in claim 17, wherein the tampon body is multi-lobed, with longitudinally extending lobes that enlarge towards the proximal end of the tampon body.

19. A tampon as claimed in claim 17, wherein the tampon body panels are assembled together by being overlaid in a stack each at a different radial orientation to a neighbouring panel and initially in a form resembling a windmill but that is then folded forwardly and the panels held together.

20. A tampon as claimed in claim 18, wherein the tampon body panels are assembled together by being overlaid in a stack each at a different radial orientation to a neighbouring panel and initially in a form resembling a windmill but that is then folded forwardly and the panels held together.

21. A tampon adapted for use by women who suffer from clotted menstrual blood flow, wherein the tampon has a body comprising an elongate mass of compressed absorbent material with a proximal end and a distal end and wherein the proximal end of the tampon is formed substantially enlarged with a greater lateral spread than the distal end of the tampon prior to wetting, wherein the tampon is housed in a tampon applicator for comfortable insertion, the applicator being of uniform external width/diameter or tapered laterally/radially inwardly towards the proximal end while the applicator internal chamber width/diameter is sized to accommodate the large head/proximally enlarged shape of the tampon proximal end.

22. Use of an adapted tampon as a treatment for women who suffer from clotted menstrual blood flow, wherein the tampon has a body comprising an elongate mass of compressed absorbent material with a proximal end and a distal end and wherein the proximal end of the tampon is formed substantially enlarged with a greater lateral spread than the distal end of the tampon prior to wetting, and has a longitudinal cleft and/or a crater or concavity at the proximal end to capture and hold viscous or gelatinous menstrual fluid.

23. Use of an adapted tampon as a treatment as claimed in claim 22, wherein the crater or concavity deepens as a void/hollow core through the tampon to capture and hold viscous/gelatinous menstrual fluid, the tampon being absorbent through its exterior.

24. A tampon adapted for use by women who suffer from clotted menstrual blood flow, wherein the tampon has a body comprising an elongate mass of compressed absorbent material with a proximal end and a distal end and wherein the proximal end of the tampon is formed substantially enlarged with a greater lateral spread than the distal end of the tampon prior to wetting, and has a longitudinal cleft in a side and/or a crater or concavity at the proximal end to capture and hold viscous or gelatinous menstrual fluid, wherein the tampon body has a substantially trapezoidal shape as viewed in side elevation and/or is frusto-conical form.
